(19) United States
(12) Patent Application Publication
(10) Pub. No.: US 2013/0345295 A1
(43) Pub. Date: Dec. 26, 2013

(54) TARGETED ONCOLYTIC ADENOVIRUS FOR TREATMENT OF HUMAN TUMORS, CONSTRCUTION METHOD AND APPLICATION THEREOF

(71) Applicant: Beijing Bio-Targeting Therapeutics Technology Inc., Beijing (CN)

(72) Inventors: Yaohe WANG, Zhengzhou (CN); Guozhong JIANG, Zhengzhou (CN); Pengju WANG, Zhengzhou (CN); Dongling GAO, Zhengzhou (CN); Nick LEMOINE, London (GB)

(73) Assignee: Beijing Bio-Targeting Therapeutics Technology Inc., Beijing (CN)

(21) Appl. No.: 14/016,149

(22) Filed: Sep. 2, 2013

Related U.S. Application Data
(63) Continuation-in-part of application No. PCT/CN2012/071754, filed on Feb. 29, 2012.

(30) Foreign Application Priority Data
Mar. 2, 2011 (CN) 201110050046.0

(51) Int. Cl. A61K 31/711

(52) U.S. Cl. CPC A61K 31/711
USPC 514/44 R; 435/320.1; 435/6.12

(57) ABSTRACT

An oncolytic adenovirus vector and its potential application in cancer treatment and vaccination. The inventive vector (named Ad-TD-hIL12) is derived from the human adenovirus group C type 5, more particularly including deletion of three adenovirus genes E1A-CR2, E1B19K and E3gp-19K, and a fused cDNA sequence of p35 and p40 subunit of human IL12 placed under the control of the E3gp-19K promoter. The invention also includes a method to construct the triple gene-deleted vector (Ad-TD). The Ad-TD-hIL12 and Ad-TD-shIL12 (with a short p40 sequence of human IL 12) vectors can be used as targeted, genetically engineered agents for treatment of various solid tumors, via not only intratumoral injection, and also in intraperitoneal injection, without causing significant side effects, showing a superior antitumor efficacy and safety.

### CROSS-REFERENCE TO RELATED APPLICATIONS

This application is a continuation-in-part of International Patent Application No. PCT/CN2012/071754 with an international filing date of Feb. 29, 2012, designating the United States, now pending, and further claims priority benefits to Chinese Patent Application No. 201110050046.0 filed on Mar. 2, 2011. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 14781 Memorial Drive, Suite 1319, Houston, Tex. 77079.

### BACKGROUND OF THE INVENTION

1. Field of the Invention

The invention relates to the field of gene engineering, and more particularly to an oncolytic adenovirus for treating human tumors and the applications thereof.

2. Description of the Related Art

Adenovirus vector Ad-TD, a kind of tumor-targeted vector created by genetic engineering technology, is a type 5 adenovirus of which genes E1A-CR2, E1B19K, and E3gp-19K are removed, and the promoter sequence of E3gp-19K is retained. The vector has a superior anti-tumor efficacy compared to the first generation of onclytic adenovirus. Interleukin 12 (IL12) is well-known as a stimulatory factor for Natural Killer Cell and a maturation factor for Cytotoxic T Lymphocytes, and it is a cytokine heterodimer connected by disulfide bonds, and the two subunits are p35 and p40. p35 is produced by T cells, B cells, NK cells, monocytes and other cells; while p40 is mainly produced by activated monocytes and B cells. The sequences of the p35 gene and the p40 gene of human and mouse have been determined, and they have bioactivities as growth factors for NK cells and T cells both in vivo and in vitro. Further studies have shown that IL12 can effectively inhibit or completely eliminate tumors in mice. However, the half-life of IL12 in vivo is very short, so only continuous injection can maintain the therapeutic efficacy, requiring a large amount of IL-12 (1-10 μg/day). Administration of high doses of recombinant IL-12 protein always leads to severe toxicity. Currently, IL12 gene therapy using retroviral vector has been conducted in some laboratories, and its anti-tumor efficacy has been proven. However, direct utilization of IL-12 gene therapy cannot eliminate established tumors and the spontaneous metastases of tumors due to the limited expression of IL12 using the non-replicative vector.

### SUMMARY OF THE INVENTION

In view of the above-described problems, it is one objective of the invention to provide an adenovirus Ad-TD-hIL12 for targeting treatment of human tumors. The virus can be used as a genetically engineered agent for treating tumors, as the viral vector selectively replicates within tumor cells but not normal cells, and it is safe and highly efficient.

The type 5 human adenovirus vector includes an expression cassette with the p35 and p40 subunit genes encoding human IL12 and the virus can selectively replicate in tumor cells and express functional human protein IL12 after infection of tumor cells. These tumors comprise solid tumors, metastatic tumors, and diffusely spreading tumors. The viral vector can selectively amplify in tumor cells, lyse tumor cells, release large amount of tumor-associated antigens, and cooperate with the expressed cytokine human IL12 to effectively induce tumor-specific immunity, which can kill the uninfected tumor cells locally and at remote areas comprising micro-metastatic tumor cells. The highly expressed IL12 in tumor cells can also prevent neovascularization.

To achieve the above objective, in accordance with one embodiment of the invention, there is provided a tumor-targeted adenovirus vector Ad-TD-hIL12. The vector is a type 5 human adenovirus of group C, of which three intrinsic genes E1A-CR2, E1B19K, and E3gp-19K are removed (Therefore named Ad-Triple Deletion, Ad-TD) and the E3B gene that facilitates the expression of viral genes and enhances the persistence of the viruses in vivo is retained. Furthermore, the endogenous promoter of E3gp-19K is retained to drive the expression of the exogenous therapeutic gene—human IL12.

IL12 is a heterodimer comprising a p35 subunit and a p40 subunit, each subunit is encoded by its corresponding gene, and the coding DNA sequences for p35 and p40 subunits of IL12 can be obtained from human cells. The human IL12 sequence in this invention comprises the coding cDNA sequences of IL12 derived from human lymphocytes, in vitro synthesized sequences, and recombinant intact or modified coding sequences with anti-tumor efficacy. These sequences comprise but are not limited to point mutation, and internal, 5′ and/or 3′ end deletion of DNA sequence of human IL12. The homologous DNA sequence or the encoded polypeptide showing anti-tumor efficacy or the sequences inhibiting tumors that are different from the human IL12 sequence in the present invention are also included.

In a class of this embodiment, corresponding amino acid sequences for the p35 and p40 subunit genes are SEQ NO: 1 and SEQ NO: 2 respectively, corresponding nucleotide sequences are SEQ NO: 3 and SEQ NO: 4 respectively. It should be noted that, the sequences of p35 and p40 subunits optionally comprises homologous sequences or polypeptides that are different from the human IL12 sequence, but show anti-tumor efficacy or inhibit tumors, including but not limited to SEQ NO: 5 and 6.

In a class of this embodiment, the human tumors are all types of human solid tumors.

In another aspect, the invention provides a method for treating human tumors comprising administering the Ad-TD-hIL12 to a patient in need thereof.

In accordance with another embodiment of the invention, the invention provides a method for constructing the tumor-targeted oncolytic adenovirus Ad-TD-hIL12 for treating human tumors, comprising the following steps:

1) Collecting peripheral blood from healthy donor, isolating and culturing lymphocytes in the presence of phytohemagglutination (PHA), extracting RNA and preparing cDNA by reverse transcription, cloning p35 and p40 subunit cDNA of human IL 12 by PCR in the presence of primers including specific enzymatic restriction sites, linking the p35 and p40 subunit gene fragments to yield an intact hIL12 gene fragment, introducing the intact hIL12 gene fragment into a cloning vector to yield pORF-hIL12, digesting the pORF-hIL12 plasmid using two enzymes of Nco I and Nhe I, and blunting the resultant hIL12 gene fragment using T4 DNA polymerase for further cloning;

2) Digesting an adenovirus vector pAd-TD using a blunt end enzyme (SWAI, which is localized between Ad E3 6.9K and the residual region of of E3gp-19K), inserting the hIL12 gene into the region by ligation, and identifying the recombinant vectors with correct insertion by PCR; and

3) Transfecting the recombinant vector into 293 cells to produce the infectious viral vector Ad-TD-hIL12.

The tumor-targeted adenovirus Ad-TD-hIL12 in this invention has been preserved in the China Center for Type Culture Collection, the serial number for the preservation is CCTCC NO: V201031, and the preservation day is Dec. 1, 2010.

Advantages of the invention are summarized as follows:
1) The tumor-targeting adenovirus Ad-TD-hIL12 utilizes the endogenous promoter of a viral gene of the virus, three intrinsic adenovirus genes of E1A-CR2, E1B19K, and E3gp-19K are deleted, other genes in the E3 region are retained, and the promoter of the E3gp-19K gene is used to express human IL12 as a therapeutic gene. The vector and therapeutic gene can selectively amplify in tumor cells rather than normal cells by specifically targeting the commonly deregulated Rb gene and anti-apoptosis genes in human tumor cells, thereby ensuring specificity and safety. The replicated viruses can lyse tumor cells and produce high levels of IL12 protein while they replicate within tumor cells, with multiple anti-tumor effects in tumor tissues.
2) The hIL12 expressed by the Ad-TD- hIL12 can prevent neovascularization, and more importantly, it can regulate host immunity and produce a synergistic effect with the replicated Ad-TD-hIL12 in tumor cells, whereby producing specific anti-tumor immunity in organisms, killing metastatic tumor cells from remote places, and preventing recurrence of tumors. Cytological and animal tests show that the virus can selectively kill tumor cells and eradicate tumors established in immune-deficient and immune-competent animals, with an excellent profile of therapeutic efficacy and safety.
3) The tumor-targeted adenovirus Ad-TD-hIL12 in this invention can specifically target tumors and results in superior anti-tumor efficacy. The adenovirus can be used as a targeted, genetically engineered agent for treatment of tumors comprising primary solid tumors, metastatic tumors, and diffusely spreading tumors.
4) The tumor-targeted adenovirus Ad-TD-hIL12 in this invention can not only be used for intratumoral injection, but also be used for intrathoracic injection and intraperitoneal injection, all of which cause no significant side effects.
5) The tumor-targeted adenovirus Ad-TD-hIL12 in this invention is safe and highly efficient, which provides a proof of concept to translate it into clinical applications.

### BRIEF DESCRIPTION OF THE DRAWINGS

FIG. 1 shows the schematic structures of tumor-targeted adenovirus Ad-TD-hIL12, Ad-TD-gene, and d11520 in accordance with one embodiment of the invention and a structural diagram of a control virus Ad-TD-RFP;

FIG. 2 shows therapeutic efficacy of Ad-TD-hIL12 and a control virus in animals bearing larger starting tumors;

FIG. 3 shows tumor growth curves in Syrian hamsters bearing Syrian hamster-derived tumors after treatments with Ad-TD-hIL12, a control virus and dl1520 at different dosages;

FIG. 4 shows the percentage of tumor progression-free animals bearing Syrian hamster-derived tumors after treatments with Ad-TD-hIL12, a control virus and dl1520 at different dosages;

FIG. 5 shows the tumor eradication rate in Syrian hamsters bearing Syrian hamster-derived tumors after treatments with Ad-TD-hIL12, a control virus and dl1520;

FIG. 6 shows tumor growth curves in Syrian hamster bearing Syrian hamster pancreatic cancer after treatment with low dosages of Ad-TD-hIL12, a control virus and dl1520;

FIG. 7 shows the percentage of tumor progression-free animals bearing Syrian hamster-derived tumors after treatments with low dosages of Ad-TD-hIL12, a control virus and dl1520;

FIG. 8 shows the tumor eradication rates of tumors in animals bearing subcutanerous Syrian hamster-derived tumors after treatment with low dosages of Ad-TD-hIL12, a control virus and dl1520;

FIG. 9 shows induction of tumor-specific immunity in immune-competent animals bearing syngeneic tumors after treatment with Ad-TD-m/hIL12;

FIG. 10 shows therapeutic effects of Ad-TD-hIL12 at different dosages for treating peritoneally spreading pancreatic cancer (1: PBS; 2: Ad-TD-hIL12 1×10⁹ pt/time; 3: Ad-TD-hIL12 2.5×10⁹ pt/time; 4: Ad-TD-hIL12 5×10⁹ pt/time);

FIG. 11 shows a comparison of amino acid sequences of p40 and short-p40 of human IL12;

FIG. 12 shows expression of shIL12 (with a short sequence of p40) by Ad-TD-shIL12 in human tumor cells (Lane 1 indicates the expression of intact human IL12, Lane 2 indicates the expression of shIL12);

FIG. 13 shows tumor growth curves of subcutaneous tumors in immune-competent animals bearing Syrian hamster-derived tumors after treatment with Ad-TD-shIL12 and a control virus;

FIG. 14 shows a tumor growth curve of peritoneal spreading pancreatic tumors in immune-competent Syrian hamsters after treatment with Ad-TD-hIL12;

FIG. 15 shows an ascites volume curve of Syrian hamster bearing peritoneal spreading pancreatic tumors after intraperitoneal injection of Ad-TD-hIL12.

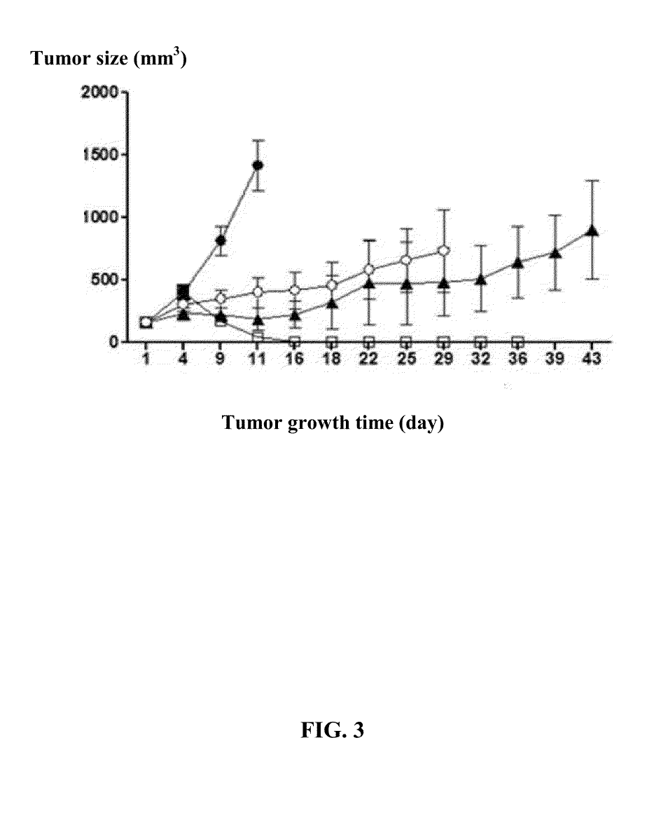

In FIGS. 3, 4, 6, and 7, the following references are used: [symbol] : PBS; [symbol] : dl1520; [symbol] : Ad-TD-RFP; [symbol] : Ad-TD-hIL12; [symbol] : Ad-TD-shIL12.

### DETAILED DESCRIPTION OF THE EMBODIMENTS

For further illustration of the invention, experiments detailing an oncolytic adenovirus for treating tumors are described below. It should be noted that the following examples are intended to describe and not to limit the invention.

### EXAMPLE 1

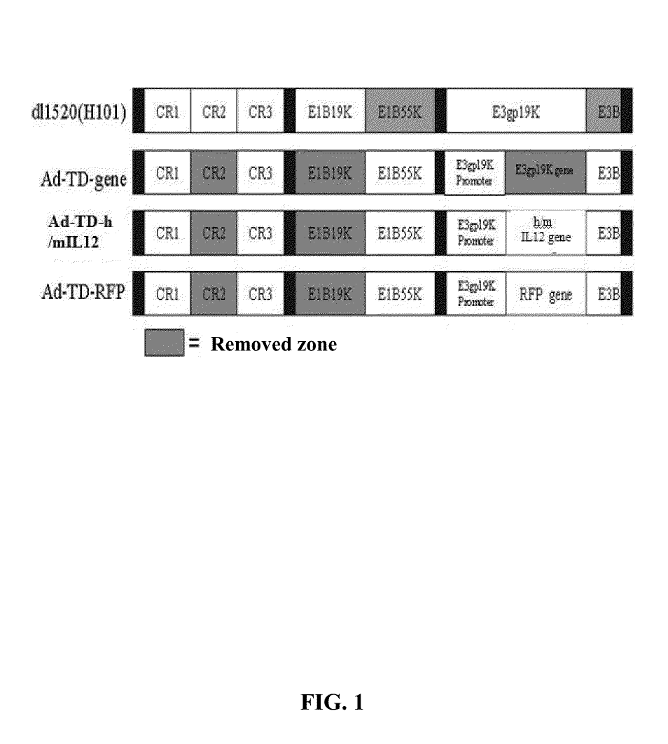

Structure of a tumor-targeted adenovirus Ad-TD-hIL12 (corresponding to human IL12 gene) and Ad-TD-mIL12 (corresponding to mouse IL12 gene), are shown in FIG. 1. The method for constructing the viral vector is described as follows:

(1) First, the DNA fragments at both sides of E1A-CR2 region to be deleted were obtained by PCR, the upstream sequence is named as left arm and the downstream sequence is named as right arm, the left arm and the right arm were ligated with a plasmid pSuperShuttle according to the virus gene sequence by genetic engineering method to construct a shuttle vector of E1A-CR2;

The adenovirus vector Ad5 and the shuttle vector of E1A-CR2 were transformed into BJ5183 for a homologous recombination at a ratio of 1: 2-10; PCR was performed to identify the positive recombinant bacteria, the recombinant plasmid was extracted and a Ad5R-CR2 viral vector comprising E1A-CR2 depletion is obtained;

(2) The shuttle vector of E1B19K is constructed by using the same method as for constructing the shuttle vector of E1A-CR2, afterwards it was recombined with the viral vector of Ad5R-CR2, and an Ad5R-CR2-E1B19K viral vector with dual depletion of E1A CR2 and E1B19K was created;

(3) PCR was carried out to amplify the sequences at both sides of the coding region of E3gp-19K gene, the left arm starts from −1087 bp and ends at 0 bp, comprising the promoter of E3gp-19K gene; the right arm starts from 1146 bp downstream the stop codon of E3gp-19K gene, the two arms were connected with enzymatic restriction sites to construct a shuffle vector which was further recombined with the viral vector Ad5ΔR-CR2-E1B19K to obtain an adenovirus vector of pAd-TD with triple depletion of three coding genes E1A-CR2, E1B19K, and E3gp-19K. In addition, pAd-TD has two SwaI restriction enzyme sites in the deleted region of E3gp 19K. Thereafter, PacI digested pAd-TD was transfected into 293 cells to produce infectious tumor-targeted adenovirus vector of Ad-TD-gene;

(4) Peripheral blood from healthy donor was collected. The lymphocytes were isolated and cultured in the presence of phytohemagglutination (PHA), the RNA was extracted and reverse-transcripted into cDNA. The primers comprising enzymatic restriction sites were used for cloning p35 and p40 subunit cDNA of human IL 12 (or short IL12 or mIL 12) by PCR, the p35 and p40 subunit gene fragments were linked by DNA ligase to yield an intact hIL12 gene fragment, which was further inserted into cloning T vector and named as pORF-hIL12 or pORF-shIL12. The plasmids were digested with Nco I and Nhe I to release the coding cDNA fragment of hIL12 or shIL12 or mIL 12 for the next step of cloning;

(5) The deletion region of E3gp-19K of the adenovirus vector of pAd-TD is digested with a blunt end enzyme (SwaI), the hIL12 or shIL12 cDNA coding fragment released from pORF-h/mIL12 vector was inserted into the enzymatic site of the viral vector of pAd-TD in accordance with the genomic sequence, and PCR was carried out to identify the recombinant vector with a correct insertion; and

(6) The recombinant vector with correct insertion was digested with PacI and the viral fragments were transfected into the 293 cells to produce the infectious viral vector Ad-TD-h/mIL12 or Ad-TD-shIL 12.

### EXAMPLE 2

### Anti-Tumor Efficacy of Tumor-Targeted Adenovirus Ad-TD-hIL12

1×10⁶ HPD1-nr cells (pancreatic cancer cells of Syrian hamsters) were subcutaneously inoculated into the right upper back of the 5-6 week old Syrian hamsters(n=7/group). When the tumors approached a volume of 160 mm³, intratumoral injection of PBS, dl1520, Ad-TD-RFP, Ad-TD-mIL12 and Ad-TD-hIL12 was carried out, 5×10⁹ pt/injection for three times, and the tumor growth and the survival of animals were monitored.

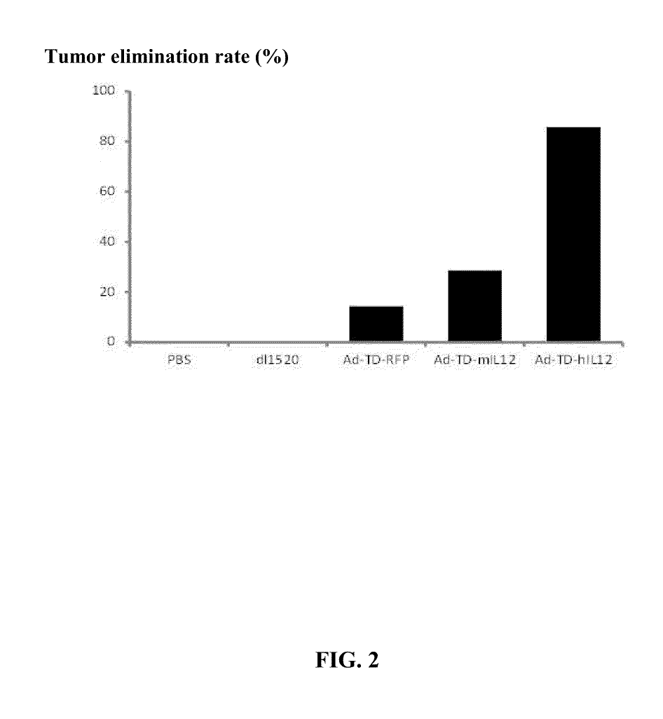

FIG. 2 shows that the percentage of animals remaining tumor-free after treatment with different viruses. Ad-TD-hIL12 resulted in 85.71% of animals remaining tumor-free whereas no animals were tumor-free in the dl1520-treated group.

### EXAMPLE 3

### Comparison of in vivo Anti-Tumor Efficacy of Ad-TD-hIL12, Control Virus, and dl1520

1×10⁶ HPD1-nr cells were subcutaneously inoculated into the right upper back of 5-6 week old Syrian hamsters(n=7/group), when the tumors approached a volume of 160 mm³, intratumoral injection of PBS, dl1520, Ad-TD-RFP, Ad-TD-mIL12 and Ad-TD-hIL12 was carried out respectively, 5×10⁹ pt/injection, once a day for a total of five times (the procedure is same as the clinical application for licensed oncolytic adenovirus H101, but the dosage is 20 times lower than the commonly used oncolytic adenovirus in Syrian hamster tumor model), and then the tumor sizes and animal survival were monitored.

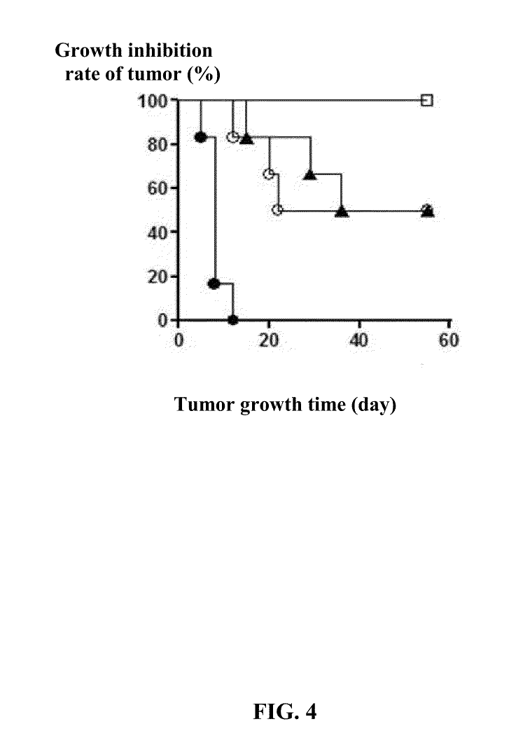

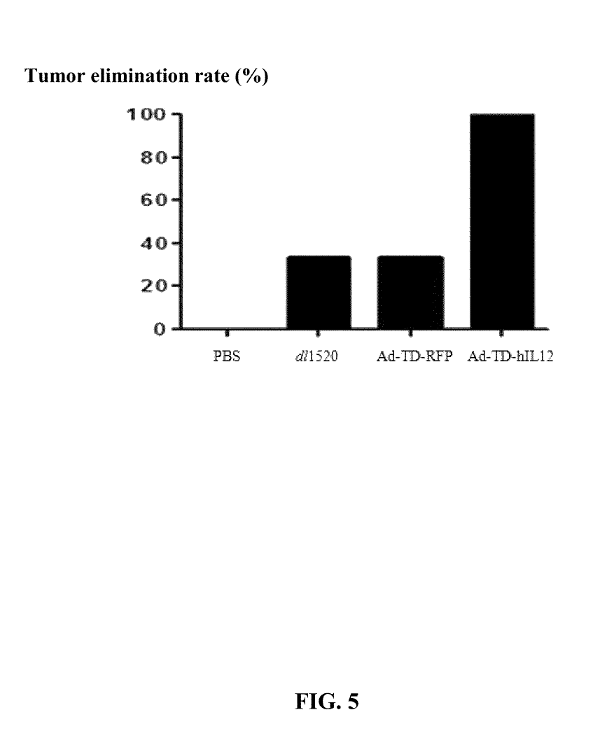

The results are shown in FIGS. 3, 4, and 5. FIG. 3 demonstrates that Ad-TD-hIL12 has superior anti-tumor efficacy compared to dl1520 and the control virus Ad-TD-RFP. FIG. 4 shows that the tumor growth in the animals treated with Ad-TD-hIL12 was the slowest. FIG. 5 shows that the tumor elimination rate in the animals after different treatments. Ad-TD-hIL12 treated group resulted in tumor eradication in 100% of animals, while the tumor elimination rates in the animals from the dl1520 group and the Ad-TD-RFP group were both 33.33%.

### EXAMPLE 4

### Comparison of in vivo Anti-Tumor Efficacy of Ad-TD-hIL12, Control Virus, and dl1520

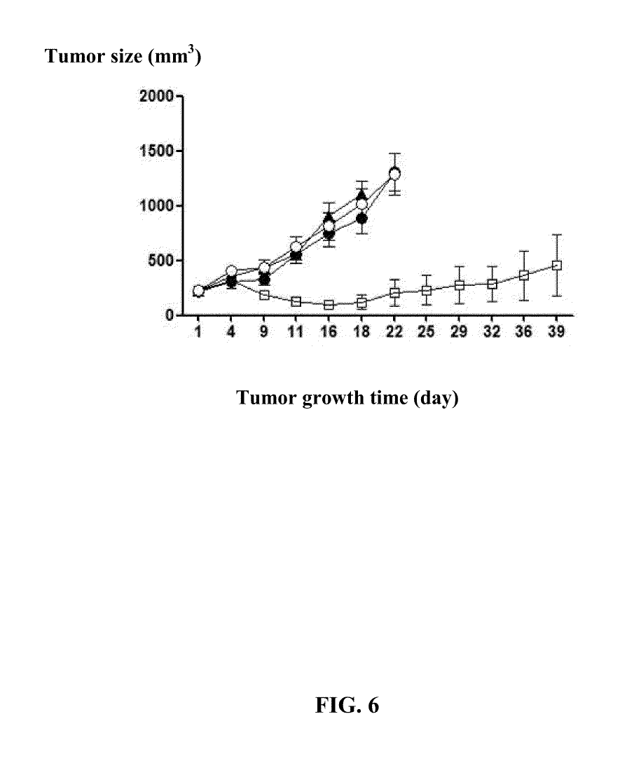

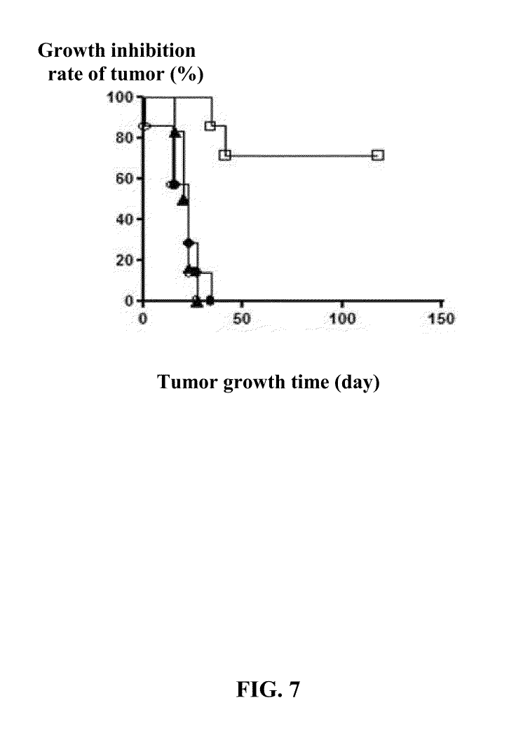

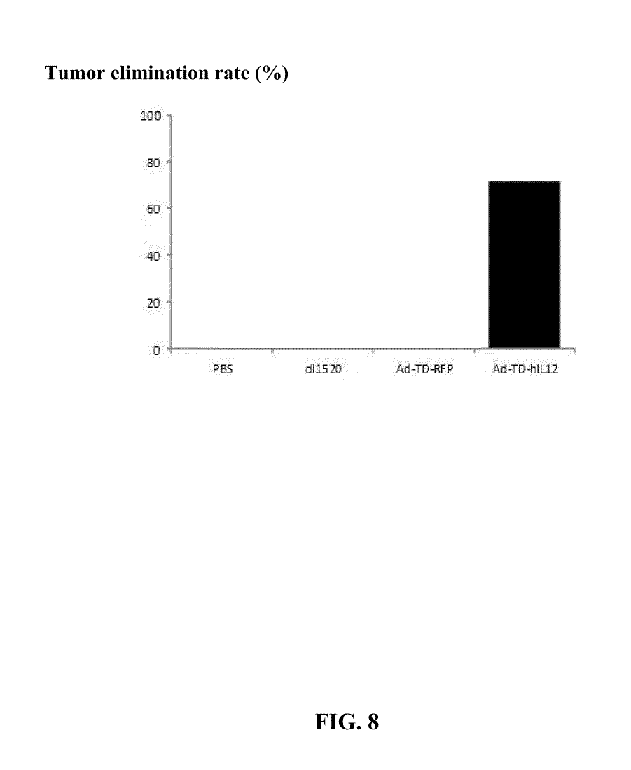

The Syrian hamster renal cancer HAK model was employed. 5×10⁶ of HAK cells were subcutaneously inoculated into the right upper back of 5-6 week old Syrian hamsters (n=7/group). When the tumors approached a relatively larger size (230 mm³), different viruses of lower dosages (1×10E⁹ pt/time, five treatments) were used, and intratumoral injection of PBS, dl1520 and Ad-TD-hIL12 was carried out. Regular measurement of the tumor sizes and the tumor elimination rates was performed. The results are shown in FIGS. 6, 7, and 8. FIG. 6 shows that at low dosage, dl1520, the control virus Ad-TD-RFP, and PBS had no significant anti-tumor efficacy, while Ad-TD-hIL12 produced a remarkable anti-tumor efficacy. FIG. 7 shows that Ad-TD-hIL12 significantly inhibited the tumor growth in the animals. FIG. 8 shows that the tumor elimination rate in the animals from the Ad-TD-hIL12 treated group is the highest (as high as 71.43%), while the tumor elimination rates in the animals from the dl1520 group and the Ad-TD-RFP group were both 0.

### EXAMPLE 5

### Induction of Tumor-Specific Immunity in Immune-Competent Animals Bearing Syrian Hamster Pancreatic Cancer After Treatment with Ad-TD-m/hIL12

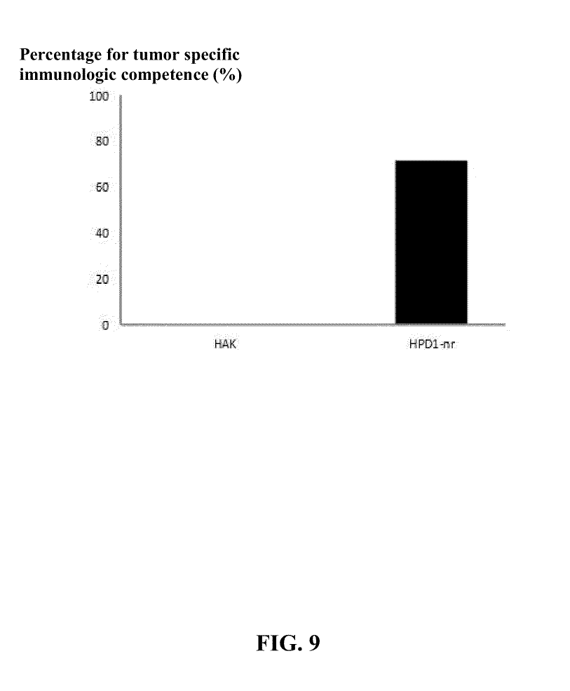

Sixty days after they became tumor-free, the animals originally bearing HPD1-nr tumors (from example 3) were subcutaneously re-challenged on the opposite flank with either 5×10⁶ of Kidney cancer HAK cells or 2×10⁶ of HPD1-nr cells. Seven days later, both types of cells produced tumors in all the animals. On the 13^{th} day, the tumors of HPD1-nr disappeared in five of seven animals and the animals remained tumor-free until at least 103 days after re-challenge. The percentage of tumor-protection against the cell type of the original tumors in the animals treated with Ad-TD-hIL12 was 71.43%. However, there was no protection against re-challenge with tumors of kidney cancer HAK cells (as shown in FIG. 9).

### EXAMPLE 6

### Therapeutic Efficacy of Ad-TD-hIL12 on Peritoneal Cavity Dissemination of Pancreatic Cancer

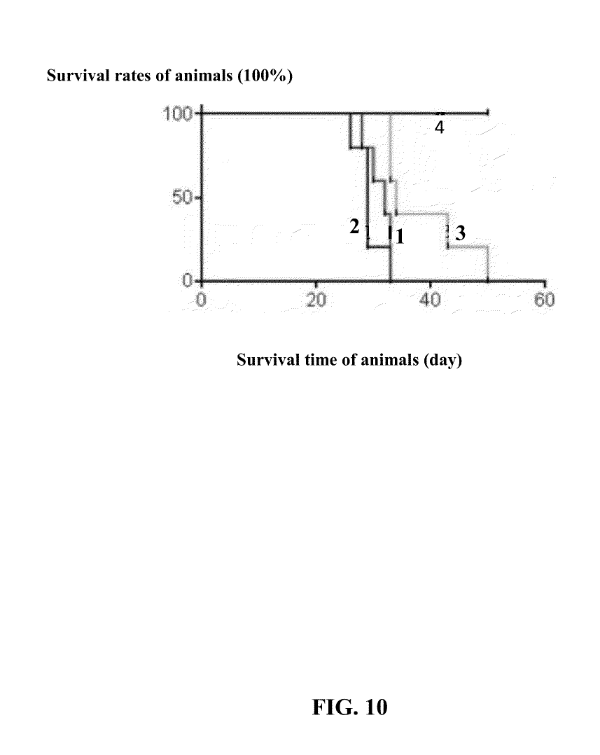

Five to six week-old Syrian hamsters (n=5/group) were intraperitoneally injected with 5×10⁶ HPD1-nr cells. The treatments were initiated 7 days later. Different reagents including PBS group, Ad-TD-hIL12 1×10⁹ pt/time, Ad-TD-hIL12 2.5×10⁹ particles and Ad-TD-hIL12 5×10⁹ particles were intraperitoneally injected once daily for five consecutive days, and the general health condition and survival were observed. Cachexia, large amount of ascites and multiple metastases were detected in the PBS-treated group and the Ad-TD-hIL12 treatment groups at a lower dose (1×10⁹ particles and 2.5×10⁹ particles) 37 days after treatment, whereas treatment with Ad-TD-hIL12 at 5×10⁹ particles significantly prolonged the survival time of the animals without adverse effects such as hepatic failure (as shown in FIG. 10).

### EXAMPLE 7

### Protocols for Construction of Tumor-Targeted Adenovirus Vector Ad-TD-shIL12

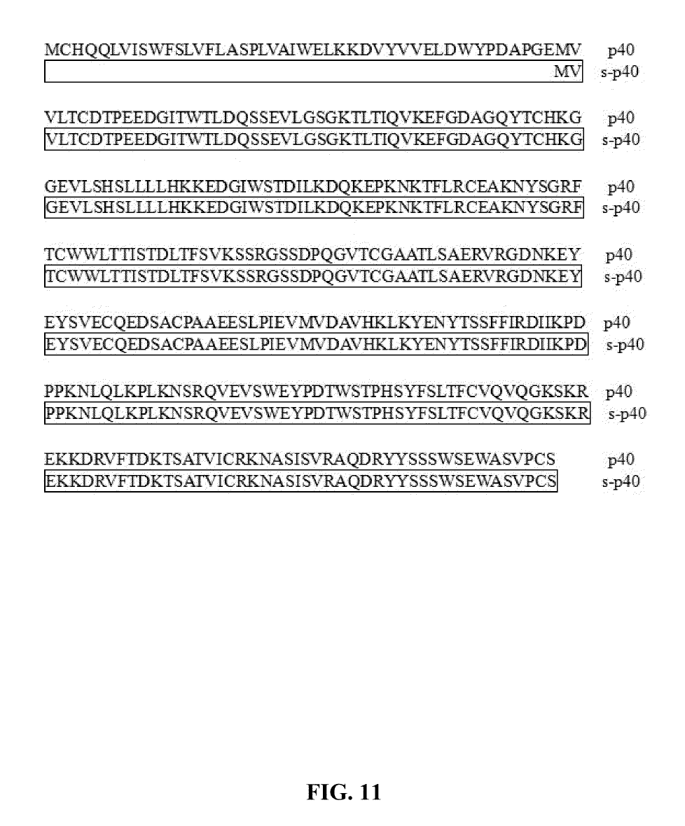

According to procedures in Example 1, a short nucleotide sequences of p40 (short-p40, s-p40) and p35 were cloned. The p35 and s-p40 subunit cDNA fragments were ligated to obtain the shIL-12 (short hIL12) gene fragment, and then pORF-shIL-12 and Ad-TD-shIL12 were constructed. The comparison in amino acid sequences of p40 and short-p40 was presented in FIG. 11, and the sequence with frame is s-p40.

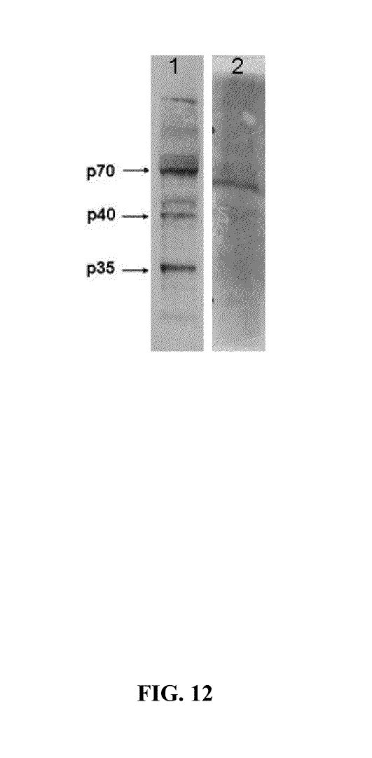

Human lung cancer A549 cells were infected with Ad-TD-shIL12. The total proteins were extracted 24 hours after infection. The expression of shIL12 was detected by Western blotting. The results show that shIL12 is expressed in A549 cells, which is lower than the intact p70 (the human IL12), as shown in FIG. 12.

### EXAMPLE 8

### Comparison of Anti-Tumor Efficacy Between Ad-TD-shIL12 and Control Virus

2×10⁶ HCPC1 cells (head and neck neoplasm of Syrian hamsters) were subcutaneously inoculated into the right upper back of 5-6 weeks old Syrian hamsters (n=7/group). When the tumors reached a volume of 160 mm³, PBS, Ad-TD-RFP, and Ad-TD-shIL12 were intratumorally injected respectively at a dosage of 5×10⁹particles once daily for a total of five times. The tumor sizes and animal survival were measured.

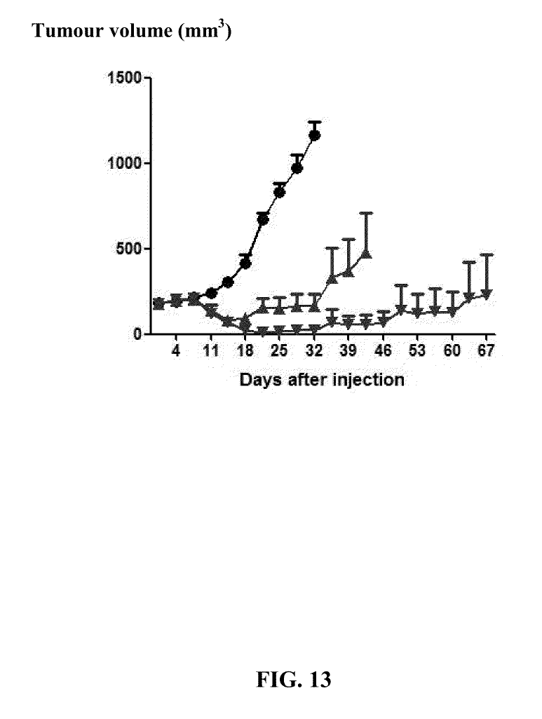

The results are shown in FIG. 13. FIG. Ad-TD-shIL12 demonstrated a superior anti-tumor efficacy than the control virus Ad-TD-RFP.

### EXAMPLE 9

### Therapeutic Efficacy of Ad-TD-shIL12 and Control Virus on Peritoneal Cavity Dissemination of Pancreatic Cancer

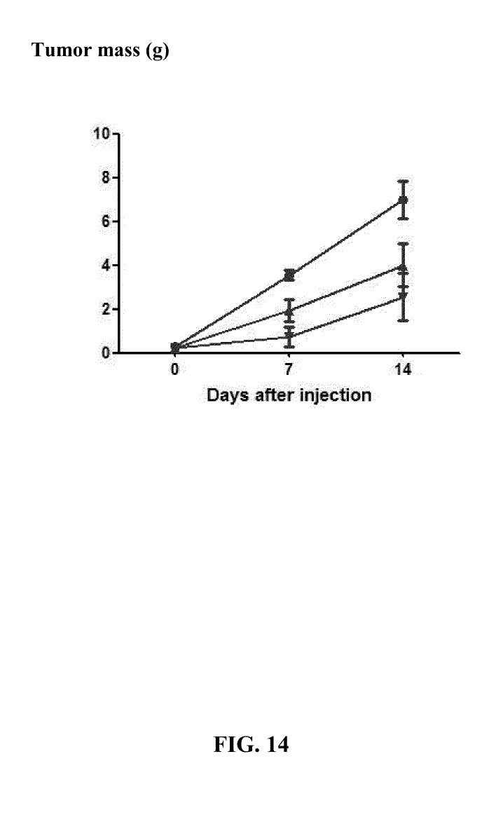

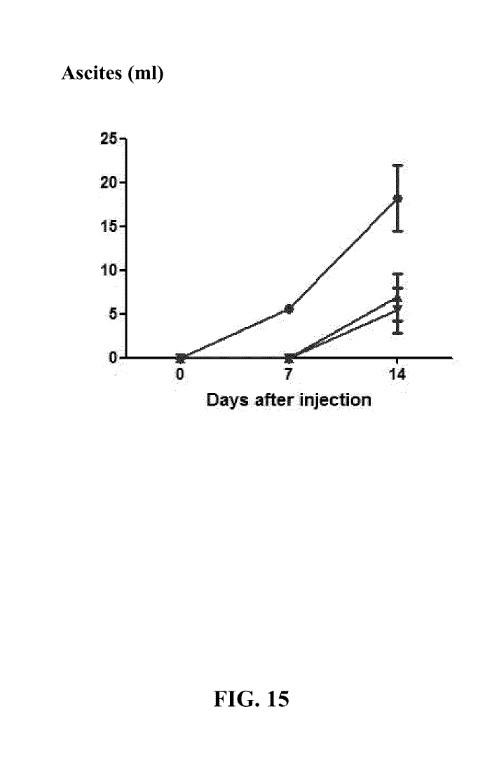

Five to six week-old Syrian hamsters (n=7/group) were intraperitoneally injected with 5×10⁶ HPD1-nr cells. The treatments were carried out 7 days later. Different reagents including PBS, Ad-TD-shIL12 and Ad-TD-RFP, were intraperitoneally injected once daily for five consecutive days, and then the survival time of the animals were observed. The virus dosage was 1×10¹⁰ pt/injection. The animals were euthanized at 0, 7 and 14 days after treatments, and the weights of tumor tissues and the ascitic volumes were measured in the peritoneal cavity. FIG. 14 reveals that only a few tumors were detected in the peritoneal cavity of the animals in the Ad-TD-shIL12 treatment group. FIG. 15 shows that only small amount of ascites was detected in the peritoneal cavity of the animals treated with Ad-TD-shIL12.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A tumor-targeted oncolytic adenovirus vector Ad-TD-hIL12 comprising deletion of three adenovirus genes E1A-CR2, E1B19K, and E3gp-19K, and a fused cDNA sequence of p35 and p40 subunit of human IL12, which is placed under the control of an E3gp-19K promoter.

2. The tumor-targeted adenovirus vector of claim 1, wherein corresponding amino acid sequences for the p35 and p40 subunit genes are SEQ NO: 1 and SEQ NO: 2, respectively, and corresponding nucleotide sequences are SEQ NO: 3 and SEQ NO: 4, respectively.

3. The tumor-targeted adenovirus vector of claim 1, wherein the sequences of p35 and p40 subunits comprise sequences obtained by point mutation, and internal, 5′ and/or 3′ end deletion of DNA sequences of human IL12, including but not limited to SEQ NO: 5.

4. The tumor-targeted adenovirus of claim 1, wherein the sequences of p35 and p40 subunits comprise homologous sequences or polypeptides that are different from the human IL12 sequence but show anti-tumor efficacy, including but not limited to SEQ NO: 6.

5. The tumor-targeted adenovirus of claim 1, wherein the tumor is a human solid tumor.

6. A method for treatment of human tumors comprising administering the oncolytic adenovirus Ad-TD-hIL12 of claim 1 to a patient in need thereof.

7. A method for construction the oncolytic adenovirus Ad-TD-hIL12 of claim 1, the method comprising the following steps:
(1) Collecting peripheral blood from healthy donors, isolating and culturing lymphocytes in the presence of phytohemagglutination (PHA), extracting RNA and preparing cDNA by reverse-transcription, cloning p35 and p40 subunit cDNA of human IL 12 by PCR in the presence of a primer comprising specific enzymatic restriction sites, linking the p35 and p40 subunit cDNA fragments to yield an intact hIL12 gene fragment, introducing the hIL12 gene fragment into a cloning T vector to make pORF-hIL12, digesting the pORF-hIL12 plasmid using two enzymes of Nco I and Nhe I, and complementing the resultant hIL12 gene fragment using T4 DNA polymerase for further use;
(2) Digesting tumor targeted adenovirus vector pAd-TD using a blunt end enzyme, inserting the hIL12 or shIL 12 cDNA coding fragment into E3gp19K region of pAd-TD in accordance with the genomic sequence, and identifying recombinant vectors with correct insertion by PCR; and
(3) Transfecting the linear Ad-TD-hIL12 or Ad-TD-shIL 12 DNA into 293 cells to produce the infectious viral vector Ad-TD-hIL12 or Ad-TD-shIL12.


SEQUENCE LISTING (6 sequences)

SEQ ID NO: 1 - protein, 198 aa, Artificial Sequence
  Fully synthetic p35
ARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 328 aa, Artificial Sequence
  Fully synthetic p40
MGHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - DNA, 597 bp, Artificial Sequence
  Fully synthetic p35
gccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 4 - DNA, 997 bp, Artificial Sequence
  Fully synthetic p40
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtag

SEQ ID NO: 5 - DNA, 865 bp, Artificial Sequence
  Fully synthetic short p40
atggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtag

SEQ ID NO: 6 - protein, 284 aa, Artificial Sequence
  Fully synthetic short p40
MVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS